(19) United States
(12) Patent Application Publication
(10) Pub. No.: US 2011/0318332 A1
(43) Pub. Date: Dec. 29, 2011

(54) IMMUNOGLOBULIN G COMPOSITION

(75) Inventors: Annie Bardat, Limours (FR); Edith Begin, Les Ulis (FR)

(73) Assignee: Laboratoire Francais Du Fractionnement Et Des Biotechnologies, Les Ulis (FR)

(21) Appl. No.: 13/139,821

(22) PCT Filed: Dec. 29, 2009
(86) PCT No.: PCT/FR2009/052714

§ 371 Date: Sep. 12, 2011

(30) Foreign Application Priority Data
Dec. 30, 2008 (FR) 0859117

(51) Int. Cl. A61K 39/395; A61P 37/00

(52) U.S. Cl. 424/130.1

(57) ABSTRACT

The invention relates to an immunoglobulin G composition comprising mannitol, glycine and a nonionic detergent, in which the immunoglobulin G concentration is 100 g/l±20 g/l.

The invention relates to an immunoglobulin G composition comprising mannitol, glycine and a nonionic detergent.

Many pathologies are currently treated with immunoglobulin G (IgG) compositions. Examples that may be mentioned include primary immune deficiencies with an antibody production defect, Kawasaki's disease, immune thrombocytopaenic purpura of children and adults, secondary immune deficiencies with an antibody production defect, in particular chronic lymphoid leukaemia or myeloma associated with repeat infections, HIV infection in children associated with bacterial infections, multifocal motor neuropathies, Guillain-Barré syndrome, chronic or severe acute parvovirus B19 infections, acquired or constitutional immunodeficiency, cortico-resistant dermatomyositis, acute myasthenia, chronic idiopathic polyradiculoneuritis, immune thrombocytopaenic purpura, for example associated with HIV infection, stiff-person syndrome, autoimmune neutropaenia, resistant autoimmune erythroblastopaenia, acquired autoantibody anticoagulant syndrome, rheumatoid arthritis, etc.

In the course of recent years, the very great demand for IgG has given rise to extreme pressure on supplies, even going as far as shortages in Europe and the United States.

In this context, there is an increasing need to produce IgG compositions, which are usually conditioned at acidic pHs and which are injectable intravenously, for example using human plasma. With the growth of these needs for IgG, the stabilization of these intravenously injectable IgG compositions (IgGIV) for their therapeutic use and their storage is of fundamental importance.

In this regard, it is known that it is necessary to stabilize IgGIVs in order especially to avoid the formation of aggregates (oligomers and polymers) liable to activate the complement system with associated risks of anaphylactic reactions. Moreover, the presence of dimers in IgGIVs has been correlated with reductions in arterial pressure in vivo (Bleeker W. K. et al, Blood, 95, 2 000, p. 6-18 6 1). Other physicochemical degradations may also take place during the storage of IgGs, for instance, inter alia, oxidation and hydrolysis.

The stabilization of IgGs thus requires the addition of compounds, conventionally chosen from sugars and amino acids, in order not only to obtain undegraded IgG compositions suitable for therapeutic use, but also IgG compositions that show increased stability on storage.

The stabilization of lyophilized forms of protein compositions and especially of IgG by addition of specific stabilizers has been the subject of numerous studies.

Those cited in the scientific publications by M. Pikal, “Freeze-Drying of Proteins, Part 2: Formulation Selection”, Biopharm. 3(9); pp. 26-30 (1990) and by Arakawa et al., Pharm. Res., 1991, 8(3), p. 285-291 show that the addition of an excipient to protein compositions before lyophilization increases the stability during lyophilization and/or the stability of the lyophilized product during storage. Among these stabilizers, some are, however, known as being agents that precipitate proteins larger than about 100 kDa. Thus, the use of polyethylene glycol (PEG) 3000-6000 is prohibitive in the freezing phase for the lyophilization of corresponding protein compositions. Osterberg et al. (Pharm. Res., 1997, 14(7), p. 892-892) have shown the efficacy of a mixture of histidine, sucrose, a nonionic surfactant and sodium chloride for the stabilization of the lyophilized forms of recombinant factor VIII and the addition of PEG did not increase their stability. In addition, Guo et al. (Biomacromol., 2002, 3(4), p. 846-849) point out that the lyophilization of horseradish peroxidase in the presence of PEG does not make it possible to conserve its native structure. The presence of PEG therefore does not appear to be desirable.

Lyophilized IgGIV compositions are commercially available, for example under the brand names Polygam™ (American Red Cross), Gammar IV™ (Armour Pharmaceutical Company) and Venoglobulin™ I (Alpha) containing as stabilizers 2% glucose, 5% sucrose and 2% D-mannitol, respectively.

International patent application WO 97/04801 describes the effect of stabilizing lyophilized monoclonal antibody formulations (such as immunoglobulin G and E) comprising specific excipients. Among these excipients, the glycine/mannitol combination is not retained due to a lack of efficacy with regard to other combinations such as sucrose/glycine and sucrose/mannitol.

It is found, however, that stabilizers which are suitable for the lyophilized forms of IgGIVs may be totally ineffective for liquid IgGIV compositions.

Thus, the commercially available liquid IgGIV compositions comprise specific stabilizers other than those used for the corresponding lyophilized form. By way of example, liquid IgGIV compositions that contain as stabilizers 10% maltose, 0.16 to 0.24 M glycine and 5% D-sorbitol are known, respectively, under the brand names Octagam™, (Octapharma), Gamunex™ 10% (Talecris) and Venoglobulin™ (Alpha).

The varied nature of the compounds used for stabilizing IgG compositions in liquid form and in lyophilized form has led certain authors to search for identical stabilizers or stabilizing mixtures that allow IgG compositions to be stored in both forms at the same time. In this regard, recent studies have focused on the stabilization of IgGIV, Vigam-S and Vigam Liquid (brand names of the National Blood Authority, UK) compositions that are liquid and after lyophilization (Vigam-S) comprising an identical mixture of stabilizers, namely albumin and sucrose (K. Chidick et al., Vox Sanguinis, 77, 204-209, 1999). However, the Vigam Liquid solution is conditioned at an acidic pH (pH 5), which has the drawback of transforming, via hydrolysis, the sucrose into reducing sugars (fructose and glucose) that condense with the lysine amino residues of the IgGs and of albumin to give an unstable Schiff's base, which changes into Maillard products (browning of the solution). It is, of course, not satisfactory to use excipients that change in the course of storage of the IgGs since control of the reaction is not possible, once it has initiated.

Moreover, some of the stabilizers mentioned previously, such as maltose or sucrose, cannot be used without risk in the case of individuals suffering from renal insufficiency and/or diabetes.

In order to overcome the above drawbacks, the Applicant has developed a single stabilizing formulation, which can stabilize both the liquid and lyophilized forms of IgGs. A particularly efficient formulation for stabilizing immunoglobulin compositions is described in the international patent application WO 2004/091 656 filed by the Applicant. This patent application discloses a composition containing 50 g/l of IgG, 50 g/l of mannitol, 10 g/l of glycine and 50 ppm of detergent, 50 ppm of detergent corresponds to a concentration of 50 mg/l of detergent.

As for many injectable medicaments, the excipients of IgGIV compositions may induce more or less pronounced undesirable side effects. These side effects are often due to the excipients themselves, which may be responsible, for example, for allergic reactions. By way of example, when 300 ml of an IgG IV concentrate of the composition described in patent application WO 2004/091 656 is administered, the amount of excipient administered to the patient will be 15 g of mannitol, 3 g of glycine and 15 mg of detergent.

Moreover, it is known that when the immunoglobulin concentration of an IgG composition is increased, oligomers and polymers may form in the said composition. The oligomers and polymers are liable to activate the complement system with associated risks of anaphylactic reactions. These oligomers and polymers are also liable to induce hypotension in the treated patient. This is not desirable and strictly controlled from a regulatory viewpoint. To avoid the appearance of oligomers and polymers when the immunoglobulin concentration of an IgG composition is increased, it is generally necessary also to increase the concentration of excipients. This increase of the concentration of excipients allows the IgG composition to be stabilized. Specifically, the excipients have a stabilizing function and their amount is generally correlated to the amount of active principle, especially when the active principle is an immunoglobulin.

Starting from the stable composition described in WO 2004/091 656 containing 50 g/l of IgG, 50 g/l of mannitol, 10 g/l of glycine and 50 ppm of detergent, the Applicant realized, surprisingly, that:

(i) not only was it possible to obtain a stable IgG composition at a concentration of 100 g/l±20 g/l of IgG while at the same time conserving the same excipients and without increasing the concentration of the said excipients;
(ii) but in addition, it was possible to obtain a stable IgG composition at a concentration of 100 g/l±20 g/l by reducing the concentration of at least one of the excipients (glycine, mannitol or detergent).

Thus, the stable composition developed by the Applicant has two major advantages over the composition already described in WO 2004/091 656:
firstly, having a larger concentration of IgG, i.e. a larger amount of active principle for the same volume, makes it possible to administer to patients a smaller volume of the said composition. The administration time is thus significantly reduced, which is reflected by less constraint on the treated patients;
secondly, administering a smaller volume of the said composition without increasing the concentration of the said excipients, preferably while reducing the concentration of at least one of the said excipients, is reflected by a large decrease in the amount of excipients administered to the patient.

Consequently, for the same amount of IgG, the volume of a composition with an IgG concentration of 100 g/l will be twice as small as the volume of a composition with an IgG concentration of 50 g/l. As a result, for an identical concentration of excipients, the amount of excipients administered with the composition with an IgG concentration of 100 g/l will be twice as small as the amount of excipients administered with the composition with an IgG concentration of 50 g/l. The risk of inducing side effects linked to the excipients is thus significantly reduced.

The invention relates to an immunoglobulin G composition comprising mannitol, glycine and a nonionic detergent, characterized in that the immunoglobulin G concentration is 100 g/l±20 g/l.

In the rest of the description, the composition according to the invention comprising 100 g/l±20 g/l of immunoglobulin G may also be referred to as the “10% IgG composition”.

The composition according to the invention is characterized by an immunoglobulin G concentration of 100 g/l±20 g/l, i.e. the composition according to the invention may have an immunoglobulin G concentration of between 80 g/l and 120 g/l. Advantageously, the composition according to the invention has an immunoglobulin G concentration of 100 g/l±10 g/l, preferably 100 g/l±5 g/l, preferably 100 g/l.

Glycine, formerly known as glycocoll or aminoacetic acid, is the simplest of the amino acids. Preferably, the glycine concentration of the composition according to the invention is between 4 g/l and 10 g/l.

Mannitol or 1,2,3,4,5,6-hexanehexol (C₆H₁₄O₆) is a polyol or “sugar alcohol” similar to xylitol or sorbitol. Mannitol was chosen by the Applicant on criteria of stability at acidic pH values for the conditioning of IgG compositions, which avoids Maillard reactions on the G immunoglobulins, on pharmaceutical compatibility criteria and on criteria relating to their stabilizing action on immunoglobulin compositions in liquid form. The mannitol concentration that is sufficient to stabilize the composition according to the invention is less than or equal to 50 g/l. Preferably, the mannitol concentration of the composition according to the invention is between 20 g/l and 50 g/l. All forms of mannitol may be used.

A suitable nonionic detergent used in the composition according to the invention is advantageously chosen from Tween®80 or polysorbate 80 (polyoxyethylene sorbitan monooleate), Tween®20 (polyoxyethylene sorbitan monolaurate), Triton® X100 (octoxynol-10) and Pluronic®F68 (polyethylenepolypropylene glycol). Preferably, Tween®80 or Triton® X100 are used. The nonionic detergents may also be combined. Preferably, the detergent is present at a concentration of between 20 and 100 mg/l, preferably between 30 and 60 mg/l, preferably between 40 and 60 mg/l and preferably between 40 and 50 mg/l. Preferably, the detergent is polyoxyethylene sorbitan monooleate (polysorbate 80).

According to one preferred embodiment, between 30 and 60 and preferably between 40 and 50 mg/l of polysorbate 80 is used, preferably 50 mg/l.

In one preferred embodiment, the composition of the invention comprises, or is preferably formed from:
100 μl of IgG
32 g/l of mannitol
7 g/l of glycine
50 mg/l of polyoxyethylene sorbitan monooleate (polysorbate 80).

Preferably, the composition of the invention has a pH of 4.6±0.2.

The 10% IgG composition according to the invention may comprise, besides mannitol, glycine and a nonionic detergent, at least one other additive. This additive may be either a compound chosen from the various categories of stabilizer conventionally used in the technical field of the invention, such as surfactants, sugars and amino acids, or an excipient added to the formulation in order, for example, to adjust its pH, its ionic strength, etc. Alternatively, the 10% IgG composition according to the invention does not comprise any excipients other than the said mannitol, glycine and nonionic detergent. Such a 10% IgG composition formed exclusively from these three compounds according to the invention has the advantage of offering good stabilization of the 10% IgG compositions and a reduction of the preparation times and costs at the industrial scale by virtue of the presence of a minimum effective number of excipients and also the presence of a minimum effective amount of excipients.

The composition according to the invention is advantageously in liquid form.

In the context of the invention, liquid IgG compositions mean aqueous solutions of compositions of polyclonal IgGs, obtained directly by fractionation of human plasma. The aqueous medium represents water for an injectable preparation (WFI) which may contain excipients that are pharmaceutically acceptable and compatible with IgGs. The IgG compositions may first undergo specific steps for inactivation/elimination of viruses, such as a detergent solvent treatment, pasteurization and/or nanofiltration. The composition according to the invention comprises IgGs that may be polyclonal or monoclonal. The IgGs may be isolated from human or animal blood or produced via other means, for example via molecular biology techniques, for example in cell systems that are well known to those skilled in the art. The composition according to the invention is particularly suited for highly purified IgGs. Advantageously, the IgGs of the present invention are obtained by fractionating human plasma. Preferred methods for fractionating human plasma are described by Cohn et al. (J. Am. Chem. Soc., 68, 459, 1946), Kistler et al. (Vox Sang., 7, 1962, 414-424), Steinbuch et al. (Rev. Franç. Et. Clin. et Biol., XIV, 1054, 1969) and in patent application WO 94/9334, and these documents are incorporated by reference in their entirety. A method for preparing an immunoglobulin G composition is also described in patent application WO 02/092 632, which is incorporated by reference in its entirety.

The 10% IgG composition of the invention in liquid form and/or in lyophilized form may also be for therapeutic use and especially for intravenous, parenteral or subcutaneous injection. The 10% IgG composition of the invention, in liquid form after storage for a period of 6 months at 5° C. or at 25° C., has a polymer content well below the standards set by the European Pharmacopoeia (3%), advantageously less than about 0.3%.

The composition of the invention may be a pharmaceutical composition, i.e. a composition suitable for therapeutic use.

The examples and figures below illustrate the invention without, however, limiting its scope.

### KEY TO THE FIGURES

FIG. 1 is a graph showing the measurement of the turbidity on stressed and unstressed test compositions.

FIG. 2 is a graph showing the percentage of anti-HBs activity of batches over 6 months of stability at 2 storage temperatures relative to T0.

FIG. 3 is a graph showing the anti-complement activity of solutions after agitation stress or no stress (NS) as a function of the added dose of detergent.

### EXAMPLES

### Example I

### Preparation of the 10% IgG Test Compositions

An IgG composition was obtained according to the method developed by the Applicant in international patent application WO 2007/077 365 or WO 02/092 632. This composition, containing about 100 g/l of IgG (10% IgG), is adjusted to a pH of between 4.6 and 4.8.

To this 10% IgG composition are added mannitol, glycine and polysorbate 80 alone or as a mixture in the concentrations stated in Table 1.

TABLE 1
Characteristics of the test solutions

| Excipient | | | |
|---|---|---|---|
| composition | Composition F1 | Composition F2 | Composition F3 |
| IgG (g/l) | 100 | 100 | 100 |
| Glycine (g/l) | 22.5 | 7 | 7 |
| Mannitol (g/l) | 0 | 32 | 32 |
| Polysorbate 80 | 0 | 0 | 40 |
| (g/l) | | | |
| final pH | 4.6 ± 0.1 | 4.6 ± 0.1 | 4.6 ± 0.1 |

### Example 2

### Stresses Applied to the Compositions

The compositions of Example 1 (F1, F2 and F3) are then subjected to various thermal, agitation and oxidation stress tests.

The thermal stress is performed according to the publication of P. Fernandes et al., Vox Sanguinis, 1980, 39, p. 101-112. In summary, samples of 5 ml of test solution are placed in 10-ml crimped glass flasks and are then heated on a water bath at 60° C. for 2 hours.

The agitation stress is performed as described in the publication from H. Levine et al., Journal of Parental Science & technology, 1991, vol. 45, No. 3, p. 160 165. Thus, samples of 5 ml of test solution are placed in 10-ml crimped glass flasks protected from light, and each flask is then placed laying down in an IKA Vibrax XR agitator (obtained from Fisher Scientific, France), and is then agitated at 500 rpm for 18 hours at room temperature.

The oxidation stress is performed on samples of 10 ml of test solution placed in 30-ml glass flasks. Hydrogen peroxide (H₂O₂) is added to each sample so as to obtain a final [H₂O₂] concentration equal to 9 mM. After stoppering and homogenizing the flasks, they are incubated for 1 h at 25° C.

### Example 3

### Measurement of the Turbidity

Each of the compositions of Example 1 (F1, F2 and F3) is or is not subjected to a stress as defined in Example 2. A turbidity measurement is performed on each sample. The lower the measured turbidity values, the more stable the IgG solutions to the applied stress.

The various measurement results obtained after application of the various preceding stresses are given in Table 2.

TABLE 2
Turbidity (NTU*)

| Test | Before | After agitation | After thermal | After oxidation |
|---|---|---|---|---|
| solution | stress | stress | stress | stress |
| F1 | 3.0 | 11.9 | 16.2 | 4.1 |
| F2 | 2.8 | 12.4 | 13.8 | 4.5 |
| F3 | 2.7 | 2.7 | 9.5 | 2.7 |

*NTU: Normalized Turbidity Units

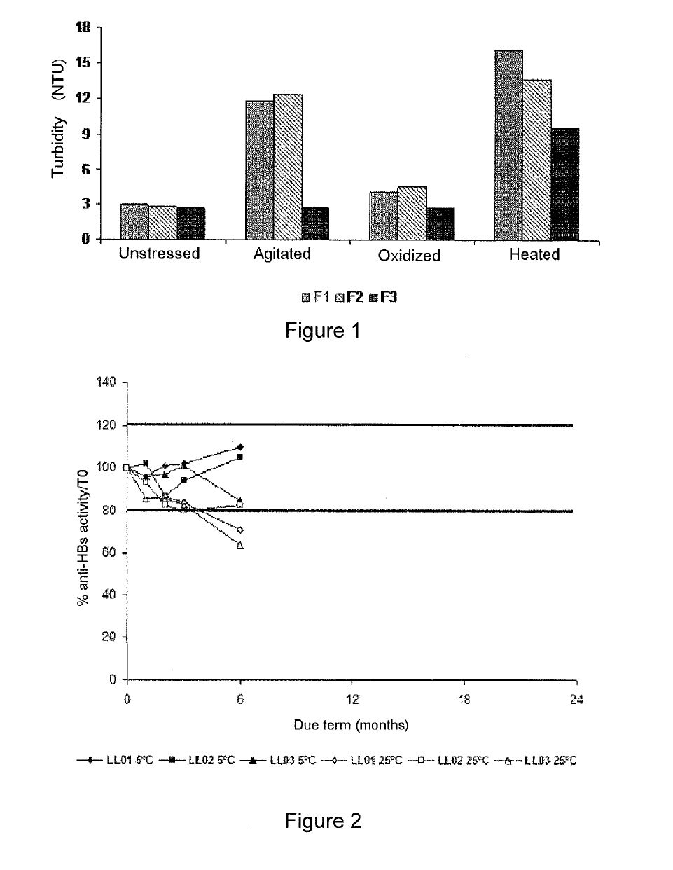

The results show that the turbidity of composition F3 is the only one that does not change after the agitation and oxidation stresses. It is also the lowest value after the thermal stress (FIG. 1).

### Example 4

### Measurement of the Anti-Complement Activity (ACA) (Method 2.6.17 of the European Pharmacopoeia)

The stressed or unstressed compositions F1, F2 and F3 are subjected to the ACA test (Method 2.6.17 of the European Pharmacopoeia) before and after each stress as described in Example 2. This test describes the ability of the immunoglobulins to activate the complement system, an excessively powerful activation of complement possibly harming the tolerance of the product during its injection. The oxidized solutions were not given for analysis, since the presence of hydrogen peroxide perturbs the assay.

Table 3 presents the ACAs of the solutions before and after stress.

TABLE 3
ACA (%)

| Test | Before | After agitation | After thermal |
|---|---|---|---|
| solution | stress | stress | stress |
| F1 | 80 | 100 | 52 |
| F2 | 61 | 100 | 51 |
| F3 | 42 | 43 | 51 |

Only formulation F3 is compliant with the standard of the European Pharmacopoeia before and after agitation. Formulations F1 and F2 have a non-compliant ACA in all the cases studied.

### Example 5

### Visual Aspect

Each of the compositions of Example 1 (F1, F2 and F3) is or is not subjected to a stress as defined in Example 2. The visual aspect of each of the samples is then observed through a pharmacopoeia inspector (Method 2.9.20 of the European Pharmacopoeia). The visual aspect makes it possible to detect the presence of large particles in the composition. The appearance of such particles reflects denaturing of the protein solution.

TABLE 4
Visual aspect

| | | After | After | |
|---|---|---|---|---|
| Test | Before | agitation | thermal | After |
| solution | stress | stress | stress | oxidation |
| F1 | No aggregates | Aggregates | Aggregates | No aggregates |
| F2 | No aggregates | Aggregates | Aggregates | No aggregates |
| F3 | No aggregates | No | No | No aggregates |
| | | aggregates | aggregates | |

Only formulation F3 has no visible aggregates in all the cases studied and withstands agitation and oxidation without any apparent modification.

### Example 6

### DLS (Dynamic Light Scattering) Measurement

Each of the compositions of Example 1 (F1, F2 and F3) is or is not subjected to a stress as defined in Example 2. The samples are then analysed in a machine that measures the size of the particles by dynamic light scattering (Malvern nanosizer). The machine measures the intensity of the Stokes radiation emitted by the particles between 0.6 nm and 6 μm in size. The particles>100 nm correspond to protein aggregates. Table 4 shows the intensity recorded for each sample for particle sizes>100 nm (and <6 μm).

TABLE 5
DLS: % intensity of particles of size >100 nm

| | | After | After | |
|---|---|---|---|---|
| Test | Before | agitation | thermal | After |
| solution | stress | stress | stress | oxidation |
| F1 | 0 | 40* | 49* | 0 |
| F2 | 0 | 46* | 77* | 3 |
| F3 | 0 | 6 | 78 | 0 |

*It should be noted that the values obtained for F1 and F2 with the agitation stress and the thermal stress are erroneous since they do not take into account the particles >6 μm (upper limit of the particles that may be detected) present in the samples. These particles >6 μm are numerous and can be seen with the naked eye (see Example 4).

Only the oxidized solutions and the agitated formulation F3 are virtually identical to the unstressed solutions.

### Example 7

### Stability of the Compositions (6 Months)

To study the stability of an IgG formulation according to the invention at a 10% concentration, 3 laboratory batches (LL01, LL02 and LL03, derived from different 5% IgG batches) were manufactured and placed under stability conditions at 5° C. and 25° C. for a period of 6 months.

The formulation of the batches is virtually as defined in composition F3 of Example 1, i.e. a final protein concentration of 100 g/l, mannitol concentration of 32 g/l, glycine concentration of 7 g/l and polysorbate 80 concentration of 40+5 mg/l.

The turbidity and ACA measurements are performed and the visual aspect is evaluated on each of the batches as described in the preceding examples.

Four other parameters are also measured over time: the assay of the anti-hepatitis B surface antigen antibodies (anti-HBs activity) according to the European Pharmacopoeia (2.7.1), the integrity of the Fc function according to the European Pharmacopoeia (2.7.9), the assay of the prekallikrein activator (pKa) and of kallikrein, and the assay of the molecular size distribution (MSD).

### Results:

Turbidity, ACA, Integrity of the Fc Function, Contents of pKa and Kallikrein, and Visual Aspect for Each Batch LL01, LL02 and LL03 Over Time:

The turbidity, the aspect, the Fc function and the pre-kallikrein and kallikrein contents are stable at 5° C. and at 25° C. in the 3 batches and do not significantly change.

There is moreover no significant variation in the anti-complement activity results, which all remain within the European Pharmacopoeia standard.

Anti-HBs Activity of the Batches LL01, LL02 and LL03 Over Time:

At each due term, the percentage of anti-HBs activity is calculated relative to T0 in the following manner:

Percentage of activity (%/T0)=(100×Tx)/T0

with Tx=activity at the due term.

FIG. 2 gives a representation of the results obtained as a percentage of anti-HBs activity over the 6 months of stability at 2 storage temperatures.

The anti-HBs activity of the solutions in terms of stability at 25° C. decreases from 1 month in the 3 batches to become non-compliant to the set internal specifications (±20%) for LL01 and LL03, whereas the anti-HBs activity does not significantly change at 5° C. The phenomenon of lowering of the anti-HBs activity is comparable to that observed on 5% IgG and is therefore not linked to the Ig concentration.

MSD:

Despite a slight increase over time, especially at 25° C., the contents of polymers and fragments remain within the warning limits (<1% for the polymers and <3% for the fragments).

At 6 months, the content of dimers is in the region of 10% and also remains within the set warning limits (<13%).

In conclusion, the three laboratory batches of 10% IgG are stable for 6 months at 5° C. and 25° C. as regards the studied parameters. Nevertheless, a decrease in anti-HBs activity at 25° C. is observed, which appears to be correlated with the increase in the content of fragments.

### Example 8

### Optimum Detergent Dose

A quantitative study of detergent is performed to determine the optimum dose to ensure the stability of the product.

The study is performed on solutions containing 100 g/l of protein formulated with 7 g/l of glycine, 32 g/l of mannitol and increasing doses of polysorbate 80 from 0 to 100 mg/l in increments of 10 mg/l (i.e. a total of 10 tested samples). The pH of these solutions is adjusted to 4.6.

The turbidity and ACA measurements are performed and the visual aspect is evaluated on each sample as described in the preceding examples.

Turbidity and Visual Aspect:

Only the formulation without polysorbate 80 shows a modification of its visual aspect after agitation and very high turbidity: aggregates appear.

ACA:

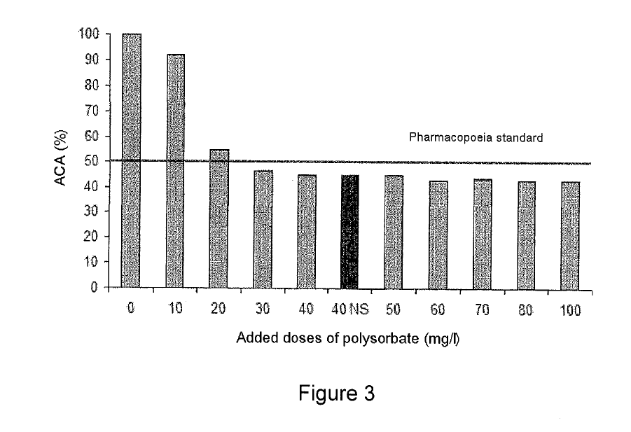

The results show that the anti-complement activity (ACA) is not compliant with the European Pharmacopoeia standard for the samples containing a 10 and 20 mg/l dose of polysorbate 80 (FIG. 3).

A lower limit of 30 mg/l of polysorbate is thus adopted since the anti-complement activity remains compliant for concentrations above 30 mg/l.

Between 40 and 100 mg/l of polysorbate 80, the anti-complement activity, and all the other parameters tested, remain constant.

### Example 9

### Stability of a Formulation Containing 50 mg/l of Polysorbate 80

Three batches of the following formulation were prepared:
100 g/l of IgG
32 g/l of mannitol
7 g/l of glycine
50 mg/l of polyoxyethylene sorbitan monooleate (polysorbate 80).

The pH is adjusted to 4.6±0.2.

The stability of these three batches was tested at 5° C. and at 25° C., for 18 months.

To do this, the following parameters were controlled.

TABLE 6

| Analyses | Expected values |
|---|---|
| Aspect of the | Clear or slightly opalescent, |
| solution | colourless or slightly yellow, |
| | without visible particles |
| pH | 4.6 ± 0.2 |
| Turbidity (NTU) | No change/T0 |
| Polymers (%) | <1.0% |
| Dimers (%) | <13.0% |
| Monomers (%) | NA |
| Fragments (%) | <3.0% |
| ACA (%) | ≦50% |
| Anti-HBs (IU/ml) | ±20% value at T0 |
| IgG (g/l) | No change/T0 |
| IgG1 (g/l) | No change/T0 |
| IgG2 (g/l) | No change/T0 |
| IgGS (g/l) | No change/T0 |
| IgG4 (g/l) | No change/T0 |
| pKa (IU/ml) | ≦35 IU/ml |
| Kallikrein (IU/ml) | <2 IU/ml |
| Function Fc (%) | ≧60% |

The three batches proved to be stable at 5° C., the measured parameters being compliant with the specifications of the European Pharmacopoeia after 18 months of storage.

The three batches also proved to be stable at 25° C., the measured parameters being compliant with the specifications of the European Pharmacopoeia after 18 months of storage, only the content of fragments showed an increase and the anti-HBs activity showed a decrease, but which remains compliant with the specifications of the European Pharmacopoeia.

1. Immunoglobulin G composition comprising mannitol, glycine and a nonionic detergent, wherein the immunoglobulin G concentration is 100 g/l±20 g/l.

2. The composition according to claim 1, wherein the glycine concentration is between 4 g/l and 10 g/l.

3. The composition according to claim 1, wherein the nonionic detergent concentration is between 20 and 100 mg/l.

4. The composition according to claim 1, wherein the mannitol concentration is between 20 g/l and 50 g/l.

5. The composition according to claim 1, in liquid form.

6. The composition according to claim 1, in which the only excipients are mannitol, glycine and the said nonionic detergent.

7. The composition according to claim 1, wherein the nonionic detergent is chosen from polyoxyethylene sorbitan monooleate, polyoxyethylene sorbitan monolaurate, octoxynol-10 and polyethylene-polypropylene glycol.

8. The composition according to claim 7, comprising from 20 to 100 mg/l of polyoxyethylene sorbitan monooleate (polysorbate 80).

9. The composition according to claim 8, comprising from 30 to 60 mg/l of polyoxyethylene sorbitan monooleate.

10. The composition according to claim 9, comprising from 40 to 50 mg/l of polyoxyethylene sorbitan monooleate, preferably 50 mg/l.

11. The composition according to claim 10, comprising:
100 g/l of IgG
32 g/l of mannitol
7 g/l of glycine
50 mg/l of polyoxyethylene sorbitan monooleate (polysorbate 80).

12. The composition according to claim 11, with a pH of 4.6±0.2.

13. The composition according to claim 1, wherein the G immunoglobulins are obtained by fractionation of human plasma.

14. The composition according to claim 2 wherein the nonionic detergent concentration is between 20 and 100 mg/l.

15. The composition according to claim 2, wherein the mannitol concentration is between 20 g/l and 50 g/l.

16. The composition according to claim 3, wherein the mannitol concentration is between 20 g/l and 50 g/l.

17. The composition according to claim 14, wherein the mannitol concentration is between 20 g/l and 50 g/l.

18. The composition according to claim 2, in liquid form.

19. The composition according to claim 3, in liquid form

20. The composition according to claim 4, in liquid form
